(19) United States
(12) Patent Application Publication
(10) Pub. No.: US 2017/0231818 A1
(43) Pub. Date: Aug. 17, 2017

(54) DEVICE FOR CUTTING TISSUE

(71) Applicant: GEUDER AG, HEIDELBERG (DE)

(72) Inventor: Volker Geuder, Heidelberg (DE)

(21) Appl. No.: 15/514,516

(22) PCT Filed: Jul. 21, 2015
(86) PCT No.: PCT/DE2015/200410

§ 371 Date: Mar. 25, 2017

(30) Foreign Application Priority Data
Sep. 26, 2014 (DE) 10 2014 219 616.7

(51) Int. Cl. A61F 9/007

(52) U.S. Cl. CPC A61F 9/00763

(57) ABSTRACT

A device for cutting and aspirating tissue from the human or animal body, preferably for use in ophthalmology, particularly for vitrectomy, for retinal peeling, etc., with an outer tube (1), and with an inner tube (11), which can move to and fro concentrically in the outer tube (1) with slight play, wherein the outer tube (1) is closed at the free end (2), and has, near the free end (2), a first and a second lateral opening (3, 4) with in each case at least one inner cutting edge (7, 8), wherein the inner tube (11) is open at the free end and there has an outer cutting edge (12), wherein the inner tube (11) has, near the free end, at least one lateral opening (13) with at least one further outer cutting edge (14), and wherein the cutting edges (7, 8, 12, 14) of the outer tube (1) and of the inner tube (11) cooperate in a cutting action during the movement of the inner tube (11), characterized in that the first and second opening (3, 4) are each designed as an asymmetrical circle, as an asymmetrical circle, a an asymmetrical ellipse or as an asymmetrical oval, and in that a thin web (6) is formed by the asymmetrical area (5) of the first and second opening (3, 4) on the outer tube (1).

The invention relates to a device for cutting and aspirating tissue from the human or animal body, preferably for use in ophthalmology, particularly for vitrectomy, for retinal peeling, etc., with an outer tube and with an inner tube which can move to and fro concentrically in the outer tube with slight play, wherein the outer tube is closed at the free end, and has near the free end, a first and a second lateral opening with in each case at least one inner cutting edge, wherein the inner tube is open at the free end, and has near the free end, an outer cutting edge, whereby the inner tube has near the free end, at least one lateral opening with at least one further outer cutting edge, and wherein the cutting edges of the outer tube and of the inner tube cooperate in a cutting action during the movement of the inner tube.

It basically deals with a surgical cutting instrument for the removal of tissue. The instrument can be used to cut the tissue on or in the body and to aspirate it from the body or out of the body. In concrete terms, it can thereby pertain to a very special cutting instrument used to break up or cut up the vitreous body in the eye and is removed/ aspirated from the eye during a standard vitrectomy. This instrument can also be used to remove blood, blood clots and changes similar to connective tissue as well as areas of the retina as part of a retinal peeling. A device of the generic type is principally suited for use in eye surgery.

We refer to DE 10 2010 050 337 A1 for the state of the art. A generic device is known from this print, whereby both the outer tube and the inner tube each comprise two lateral recesses with a dual cutting function. Similar devices are known from DE 10 2013 201 784 A1, U.S. Pat. No. 5,630,827, U.S. Pat. No. 5,474,532 and U.S. Pat. No. 5,106,364.

In the generic device according to DE 10 2010 050 337 A1, two slots with parallel cutting edges formed to each other in parallel are provided for in the outer tube. Furthermore, the free end of the inner tube is open and a lateral opening with an outer cutting edge is arranged near the free end.

In the generic device, it is problematic that depending on the tissue to be cut, quite a high cutting force must be exerted in order to cut the tissue. It is furthermore problematic that the tissue is not separated with a clean cut, but is rather punched out through the cutting edges. Particularly when using a device in ophthalmology, the tissue in the eye must be separated with a cut as clean as possible as to not damage the sensitive eye. Punching out tissue can lead to considerable irritations and wound healing disorders of the eye and must thus be avoided.

It is furthermore problematic that a constant flow of aspiration liquid and separated tissue cannot be achieved with the generic device during the movement of the inner tube. The result is therefore an alternating negative pressure—if only to a low degree—in the body or the eye that also leads to irritations on the eye, which negatively influences the healing process.

It is therefore the task of the present invention to design and upgrade a device for cutting and aspirating tissue from the human or animal body such that a clean cutting of tissue is possible with constructively simple means and that irritations on the tissue and pressure fluctuation in the body are minimized.

The above task is accomplished according to the present invention through the features in claim 1. The device in question is thereby characterized by that the first and the second opening are each designed as an asymmetrical circle, as an asymmetrical ellipse or an asymmetrical oval and that a thin web is formed by the asymmetrical area of the first and second opening.

In a method per the invention, it was first recognized that the cutting performance of the device is surprisingly improved due to the arrangement of two openings as asymmetrical circle, ellipse, or oval. Due to the circular, elliptical or oval area, the length of the outer cutting edges is maximized and a selective cutting force is thus achieved. The result is a smooth incision and punching out the tissue to be cut is thereby prevented. Furthermore, an ideal flow behavior is achieved through this area of openings while minimizing swirls of the aspiration liquid, so that a constant flow is ensured through the inner tube during the aspiration.

In a further method per the invention it was recognized that a thin web can be realized in the outer tube due to the asymmetrical design of the openings so that more cutting edges can be provided for, whereby this design does not negatively affect the flow behavior of the aspiration liquid.

To achieve an ideal cutting performance with a lowest possible cutting force, the web can run orthogonally to the longitudinal axis of the outer tube, at least partially.

To realize a most simple construction and optimization of the flow behavior, the first opening and the second opening of the outer tube can be formed axisymmetric to one another, whereby the web, at least partially, forms part of a symmetric axis. In other words, the web can be located on the mirror axis between the first opening and the second opening, at least partially. Another advantage of this embodiment is that it can be achieved by means of the axisymmetric arrangement in an especially simple way that the same flow area into the inner tube is maintained at any point in time of the movement of the inner tube.

In an advantageous arrangement, a first and a second cutting edge can be formed each on the first opening and on the second opening. A total of four inner cutting edges are thus arranged in the outer tube so that the cutting performance of the device is increased with constructively simple means.

The first cutting edge is formed in the circular, elliptical or oval area of the first and the second opening in a favorable way. Alternatively or additionally, the second cutting edge can be formed in the asymmetric area of the first and the second opening.

As already mentioned above, the outer tube comprises inner cutting edges and the inner tube comprises outer cutting edges, whereby the respective cutting edges glide past each other during the lifting motion of the inner tube, thus resulting in the cutting effect both during the fore stroke and the return stroke. From the cutting edges of the outer tube, cutting areas may extend outwards, which are defined by the material thickness of the outer tube. It is thereby feasible that the cutting edge or the cutting edges extends or extend orthogonally to the longitudinal axis of the outer tube. The cutting edges or the cutting edge can furthermore extend to the longitudinal axis of the outer tube at an angle, meaning that the cutting surface or cutting surfaces are diagonal to the longitudinal axis of the outer tube. Depending on what kind of tissue is supposed to be cut, the cutting surface can be adapted accordingly. It is thereby furthermore feasible that the cutting edges of the first and second opening arranged on the web are formed orthogonally to the longitudinal axis of the outer tube, and that the cutting surface of the first opening and the second opening running outwards from the first cutting edge are arranged diagonally to the longitudinal axis of the outer tube.

To adapt the device to the tissue to be cut in an ideal manner, the cutting surface or the cutting surfaces can be designed linear and/or concave. For example, the cutting surfaces arranged on the web can be formed linear, whereas the cutting surfaces arranged on the first and second opening are formed concave.

In a further favorable manner, the first cutting surface of the first opening running outwards from the first cutting edge and the first cutting surface of the second opening running outwards from the first cutting edge are arranged to each other in a V shape. This means that the first cutting surfaces of the first opening and the second opening can each run diagonally to the longitudinal axis of the outer tube, whereby the cutting surfaces are arranged at opposing angles to the longitudinal axis in an especially advantageous manner. The first cutting surface can therefore be at the same angle to the inner tube during a forward motion of the inner tube as the second cutting surface during a backward motion of the inner tube.

To further maximize the cutting performance, the inner tube can comprise two adjacent lateral openings near the free end, which are equipped with three or four outer cutting edges overall.

It is furthermore feasible that the outer tube has a third and a fourth lateral opening that are formed analog to the first and second opening. The third and fourth lateral opening can thereby be arranged spaced axially or in circumferential direction to the first and second opening, whereby the third and fourth opening can each be formed as an asymmetrical circle, as an asymmetrical ellipse or as an asymmetrical oval and where another thin web can be formed through the asymmetrical area of the third and fourth opening on the outer tube.

There are now different possibilities to design and further develop the teaching of the present invention in an advantageous manner. Reference must therefore be made to the claims subordinate to claim 1 on the one hand, and to the following explanation of a preferred embodiment of the invention with the drawing on the other hand. Preferred embodiments and further developments of the teaching are generally also explained in conjunction with the explanation of the preferred embodiment of the invention using the drawing. The following is shown in the drawing

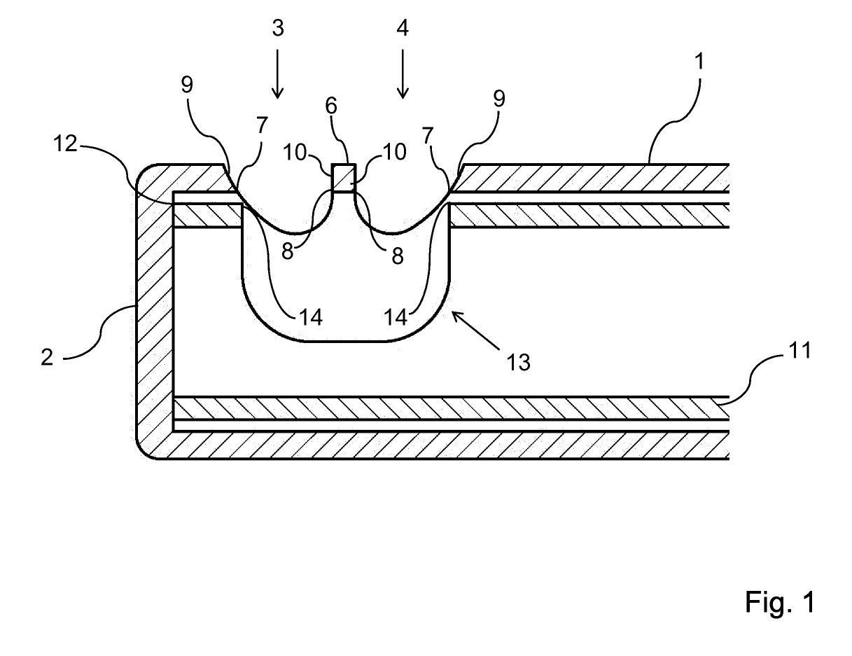

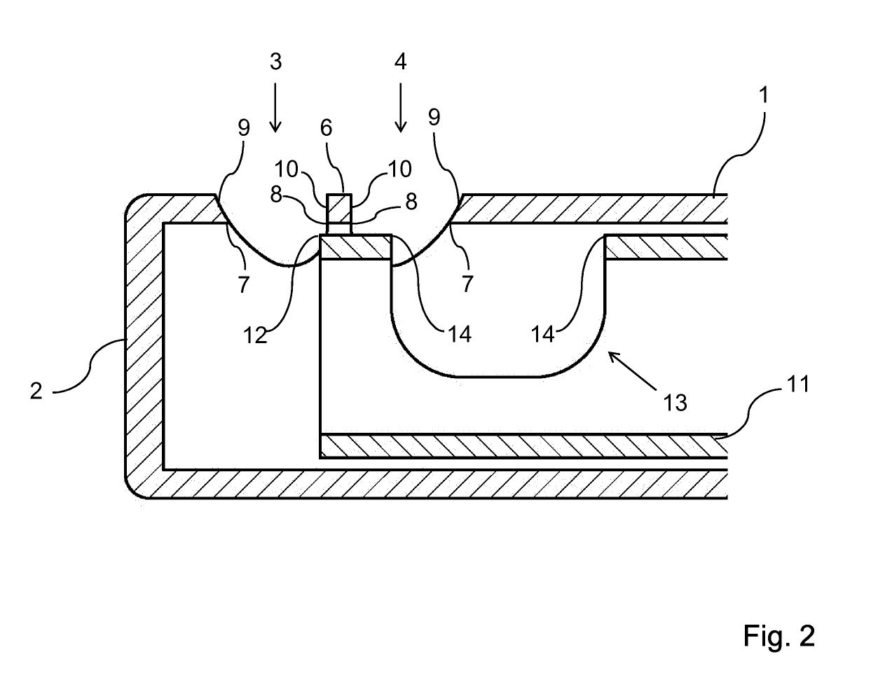

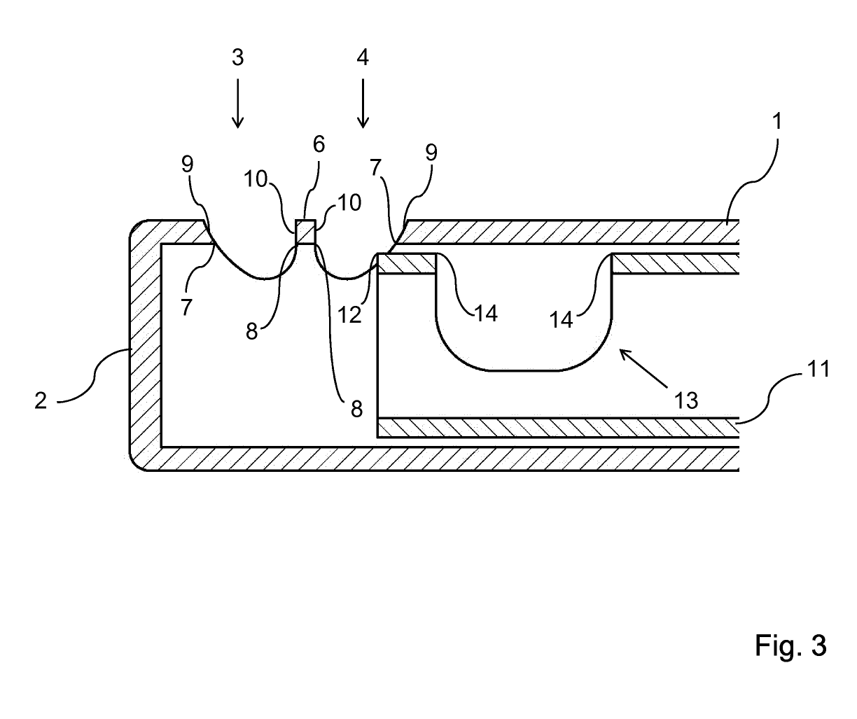

FIG. 1 to FIG. 3 in cut views, partially, an execution example of a device per the invention for cutting and aspirating tissue, whereby the figures show the movement of the inner tube in the outer tube and the openings and cutting edges each provided there,

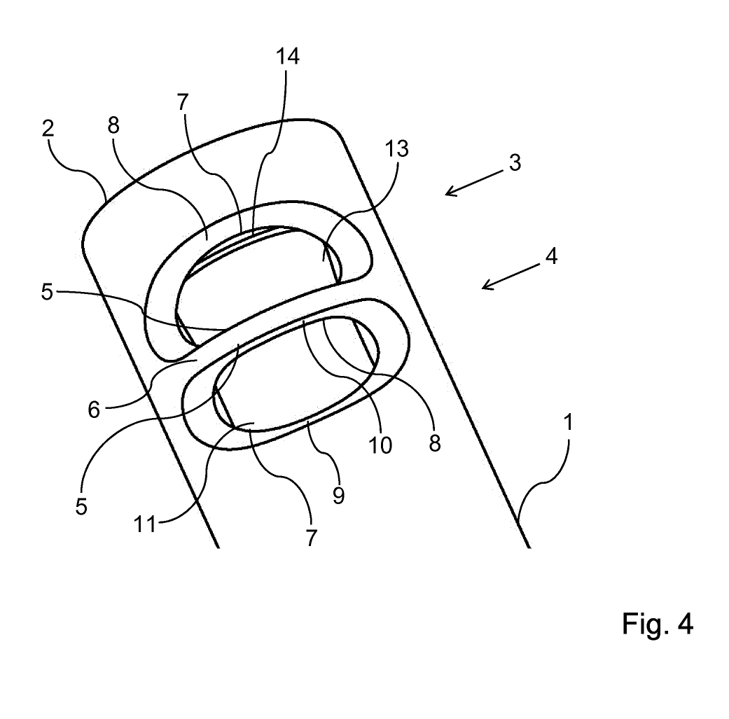

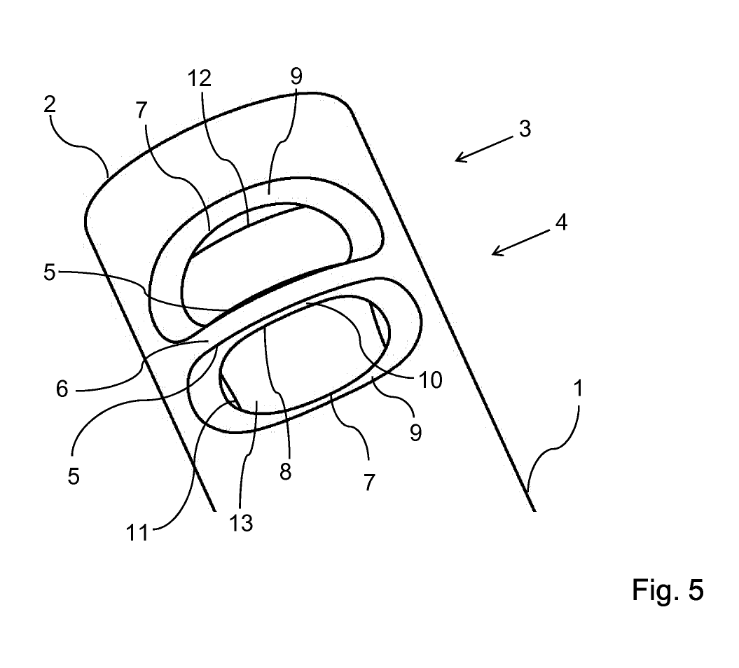

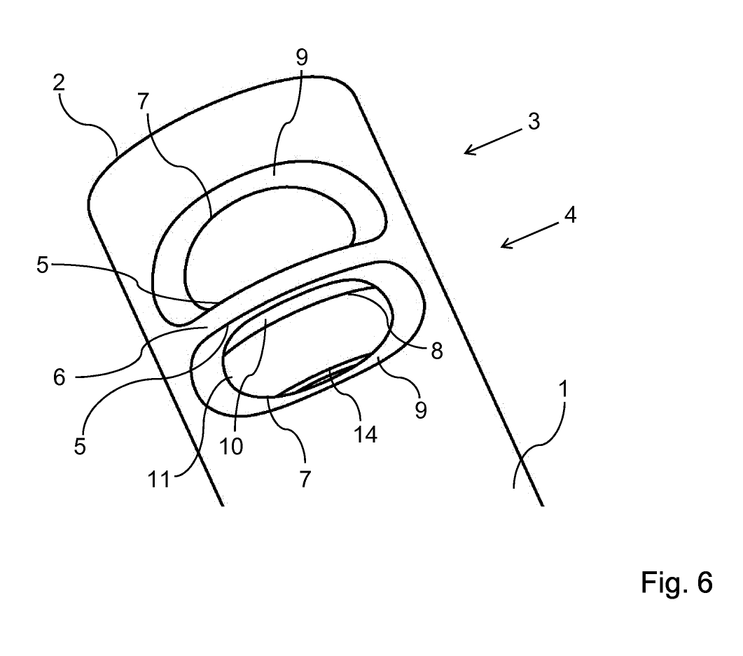

FIG. 4 to FIG. 6 in schematic views, partially, the execution example according to FIGS. 1 to 3, whereby the figures show the movement of the inner tube in the outer tube and the openings and cutting edges each provided there, and

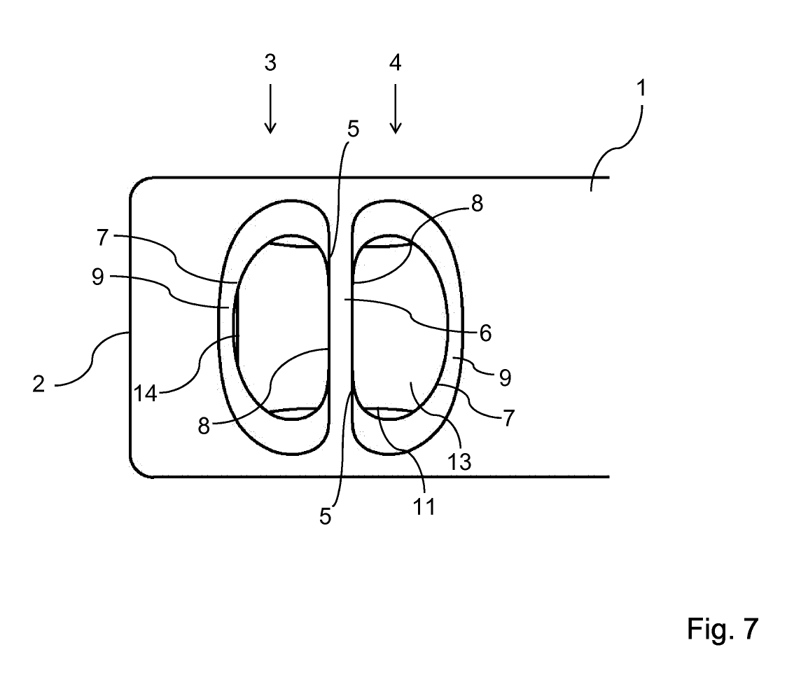

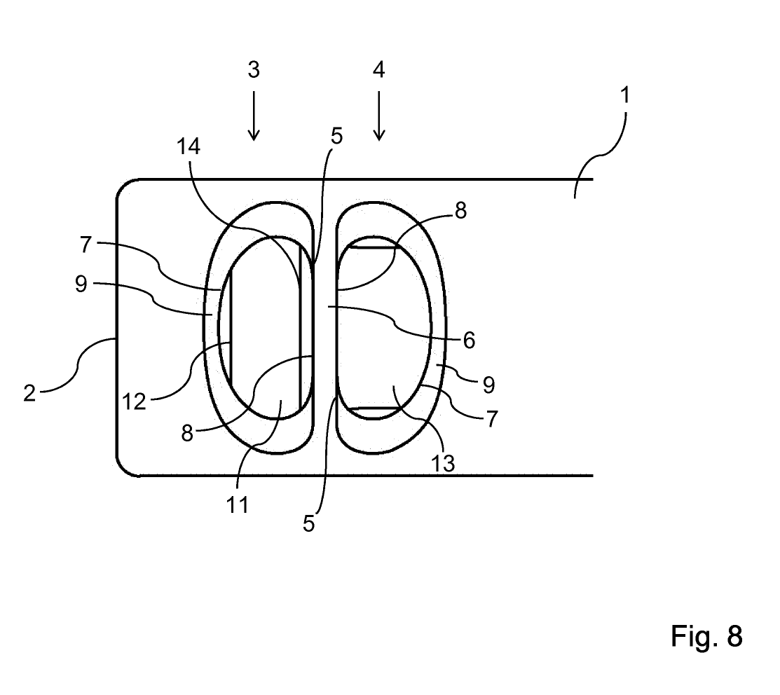

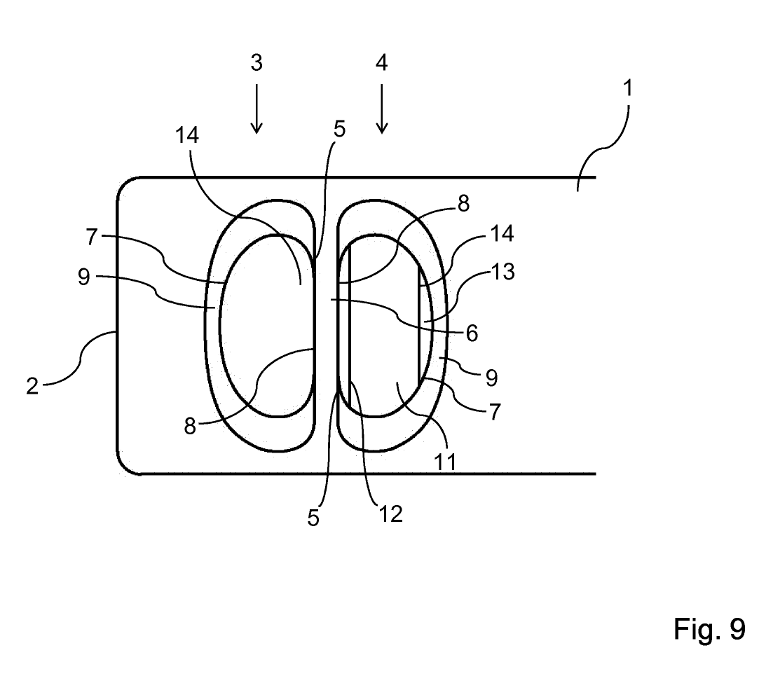

FIG. 7 to FIG. 9 in schematic views, partially, the execution example according to FIGS. 1 to 3, whereby the figures show the movement of the inner tube in the outer tube and the openings and cutting edges provided there.

FIGS. 1 to 9 show in schematic views, greatly enlarged, the work area of the device per the invention, which is a so-called vitrector for use in vitrectomy, namely a device for the removal of vitreous bodies from the human eye.

FIGS. 1 to 9 jointly show the movement and thereby the respective position of the movable parts to each other.

FIGS. 1 to 9—partially—concretely show the outer tube 1 that is closed at its free end 2. In the execution example shown in FIGS. 1 to 9, the outer tube has a first opening 3 and a second opening 4. The first opening 3 and the second opening 4 are each formed as an asymmetrical ellipse or as an asymmetrical oval. It can thereby clearly be recognized that the asymmetrical area 5 of the first opening 3 and the second opening 4 define a thin web 6 on the outer tube 4.

Furthermore, a first cutting edge 7 and a second cutting edge 8 are each formed on the first opening 3 and the second opening 4. The outer tube 1 therefore comprises a total of four cutting edges 7, 8.

Form the first cutting edges 7; first cutting surfaces 9 extend outwards, which are defined through the material thickness of the outer tube 1. The first cutting surfaces 9 are diagonal to the longitudinal axis of the outer tube 1 and are arranged to each other V-shaped. It can furthermore clearly be recognized that the first cutting surfaces 9 are formed concave.

From the second cutting surfaces 8, two more cutting surfaces 10 extend outwards, which are also defined by the material thickness of the outer tube 1. As opposed to the first cutting surfaces 9, the second cutting surfaces 10 extend orthogonally to the longitudinal axis of the outer tube 1 and are formed linear.

It is especially clearly shown in FIGS. 7 to 9 that the first opening 3 and the second opening 4 are formed axisymmetric to each other whereby the web 6 lies in the symmetry axis of the first opening 3 and the second opening 4.

An inner tube 11 is arranged in the outer tube 1 concentrically, movable alternately with slight play. FIGS. 1 to 9 indicate that the inner tube 11 is open at its frontal free end. A first cutting edge 12 is formed there accordingly. Depending on the design of the hub on the inner tube 11, the first cutting edge 12 will cut each time it glides past a cutting edge 7, 8 of the outer tube 1 aligned against the direction of movement of the inner tube. In the execution example shown in FIGS. 1 to 9, the first cutting edge 12 therefore cuts twice, namely when it is lead past a usually opposing cutting edge 7, 8 of the outer tube twice with a respectively designed hub.

The inner tube 11 can now be equipped with one or several openings. In the execution example shown here, the inner tube 11 has an opening 13 comprising two additional outer cutting edges 14, which affect a cutting process when gliding past the cutting edges 7, 8 aligned in the opposite direction.

Both when providing one single opening 13 in the inner tube 11 and also when providing several openings 13 in the inner tube, these can be dimensioned and positioned such that the same flow area has an effect, namely when the one opening 3, 4 in the outer tube 1 is opened or closed and the other opening 3, 4 in the outer tube 1 is closed or is opened. In a center position, the openings 3, 4 will thus be closed halfway through the wall of the inner tube 11 or will be opened according to the position of the cutting edge 14 of the inner tubes 11.

In terms of more advantageous embodiments of the device per the invention, reference is made to the general part of the description and the enclosed claims in order to avoid repetition.

Finally, it should be noted explicitly that the previously described execution example of the device per the invention merely serves the explanation of the claimed teaching, but that it is however not restricted to the execution example.

### LIST OF REFERENCE SIGNS

1 outer tube
2 free end (outer tube)
3 first opening (outer tube)
4 second opening (outer tube)
5 asymmetrical area
6 web
7 first cutting edge (outer tube)
8 second cutting edge (outer tube)
9 first cutting surface
10 second cutting surface
11 inner tube
12 first cutting edge (inner tube)
13 opening (inner tube)
14 cutting edge (inner tube)

1. A device for cutting and aspirating tissue from the human or animal body, preferably for use in ophthalmology, particularly for vitrectomy, for retinal peeling, etc., with an outer tube (1), and with an inner tube (11), which can move to and fro concentrically in the outer tube (1) with slight play, wherein the outer tube (1) is closed at the free end (2), and has, near the free end (2), a first and a second lateral opening (3, 4) with in each case at least one inner cutting edge (7, 8), wherein the inner tube (11) is open at the free end and there has an outer cutting edge (12), wherein the inner tube (11) has, near the free end, at least one lateral opening (13) with at least one further outer cutting edge (14), and wherein the cutting edges (7, 8, 12, 14) of the outer tube (1) and of the inner tube (11) cooperate in a cutting action during the movement of the inner tube (11),
characterized in that the first and second opening (3, 4) are each designed as an asymmetrical circle, as an asymmetrical circle, a an asymmetrical ellipse or as an asymmetrical oval, and in that a thin web (6) is formed by the asymmetrical area (5) of the first and second opening (3, 4) on the outer tube (1).

2. Device according to claim 1, characterized in that the web (6) runs, at least partially, orthogonally to the longitudinal axis of the outer tube (1).

3. Device according to claim 1 or 2, characterized in that the first and the second opening (3, 4) of the outer tube (1) is formed to each other axisymmetric, whereby the web (6) forms a part of the symmetric axis, at least in sections.

4. Device according to one of the claims 1 to 3, characterized in that a first cutting edge (7) and a cutting edge (8) each are formed on the first and the second opening (3, 4).

5. Device according to claim 4, characterized in that the first cutting edge (7) is formed in the circular, elliptical, or oval area of the first and the second opening (3, 4), and/or that the second cutting edge (8) is formed in the asymmetric area (5) of the first and the second opening (3, 4).

6. Device according to one of the claims 1 to 5, characterized in that cutting surfaces (9, 10) extend from the cutting edges (7, 8) defined through the material thickness of the outer tubes (1).

7. Device according to claim 6, characterized in that the cutting surface/ cutting surfaces (9, 10) extend orthogonally and/ or at an angle to the longitudinal axis of the outer tube (1).

8. Device according to claim 6 or 7, characterized in that the cutting surface/ cutting surfaces (9, 10) are formed linear and/ or concave.

9. Device according to one of the claims 6 to 8, characterized in that a first cutting area (9) of the first opening (3) running outwards from the first cutting edge (7) and a first cutting surface (9) of the second opening (4) running outwards from the first cutting edge (7) are arranged toward each other in a V shape.

10. Device according to one of the claims 1 to 9, characterized in that the inner tube (11) has two adjacent lateral openings near the free end, which are equipped with three or four outer cutting edges overall.

11. Device according to one of the claims 1 to 10, characterized in that the outer tube (1) has a third and a fourth lateral opening, which are formed analog to the first and second opening (3, 4).
